(19) United States
(12) Patent Application Publication
(10) Pub. No.: US 2020/0308081 A1
(43) Pub. Date: Oct. 1, 2020

(54) ROUTE FOR AROMATIC PRODUCTION FROM ISOPROPANOL AND CARBON DIOXIDE

(71) Applicant: SABIC Global Technologies B.V., Bergen op Zoom (NL)

(72) Inventors: Khalid A. Almusaiteer, Thuwal (SA); Khalid Al-Bahily, Thuwal (SA); Sang-Eon Park, Incheon (KR)

(21) Appl. No.: 15/779,683

(22) PCT Filed: Nov. 28, 2016
(86) PCT No.: PCT/IB2016/057166

§ 371 Date: May 29, 2018

(51) Int. Cl. C07C 1/22

(52) U.S. Cl. CPC C07C 1/22; C07C 15/04; C07C 2529/40

(57) ABSTRACT

A method and systems capable of producing aromatic hydrocarbons from a C3 alcohol using a catalytic metal-containing zeolite catalyst are disclosed. The method and systems can include the use of carbon dioxide as a reactant. The aromatization reactions provide product streams with high single pass aromatic selectivity of benzene, toluene and xylenes (BTX).

### CROSS REFERENCE TO RELATED APPLICATIONS

This application claims the benefit of priority of U.S. Provisional Patent Application No. 62/263,079, filed Dec. 4, 2015, which is hereby incorporated by reference in its entirety.

### BACKGROUND OF THE INVENTION

### A. Field of the Invention

The invention generally concerns a method and systems capable of catalyzing a three carbon (C₃) alcohol in the absence or in the presence of carbon dioxide (CO₂) under reaction conditions sufficient to produce an aromatic hydrocarbon containing product stream. In particular, heterogeneous zeolite catalyst compositions containing mixed metal oxides are used to catalyze the production of benzene, toluene, and xylenes (BTX) from isopropanol with and without CO₂. The disclosed methods and systems provide product streams with high single pass aromatic selectivity.

### B. Description of Related Art

Aromatic hydrocarbons are essential basic building blocks for a large number of petrochemical processes. The most important aromatic hydrocarbons are also the most simple, including benzene, toluene, and xylenes (BTX). These aromatic hydrocarbons are typically produced by catalytic reforming, coal tar processing, toluene disproportionation (TDP), and transalkylation (TA). The composition and yield of the final BTX product depends largely on the source feedstock. In recent years, the global aromatic hydrocarbons demand has far exceeded supply and demands are projected to increase for the constituents of BTX. Naphtha, a hydrocarbon feedstock used in catalytic reforming, is increasingly being substituted with shale gas (e.g., methane source) as a cheaper feedstock, which has resulted in decreased volumes of BTX produced from steam reforming. Aromatization of light hydrocarbons, in particular liquefied petroleum gas (LPG), which contains primarily methane, has grown in popularity in academic and industrial settings. Most of these investigations have focused on the use of ZSM-5 based catalysts, which have strong acidity and/or show high stability. By way of example, Applied Catalysis A: General (1999), 188, pp. 53-67 by Xu et al. discloses methane aromatization using Mo/ZSM-5 for non-oxidative aromatization with various metal active sites to promote catalytic activity and stability, however, the high activation energy of methane resulted in very low conversions and high selectivity. Other examples, including U.S. Pat. No. 3,847,793 to Schwartz et al. and U.S. Pat. No. 5,236,575 to Bennett et al., disclose the conversion of propylene to aromatics using ZSM-5 and MCM-49 type catalysts respectively, while MSc Thesis (Transformation or acetone and Isopropanol to Hydrocarbons using H-ZSM-5 Catalyst, 2009) and Vasquez (American Institute of Chemical Engineers, 2013, 59, pp. 2549-2557) disclose the conversion of isopropanol and mixed alcohols (prepared from lignocellulosic biomass using a MixAlco™ process) into aromatics using a H-ZSM-5 catalyst.

Despite all the currently available efforts produce BTX compounds from alcohols, many of the current processes suffer from poor selectivity, increased formation of by-products, and decreased BTX yields or combinations thereof.

### SUMMARY OF THE INVENTION

A discovery has been made that solves the problems associated with the production of benzene, toluene, and xylenes (BTX) from a C₃ alcohol with or without the addition of carbon dioxide (CO₂). Notably, the discovery uses heterogeneous zeolite catalyst compositions loaded with metals and metal oxides to oxidatively hydrogenate and aromatize isopropanol with high single pass aromatic hydrocarbon selectivity. In particular, the catalysts can be hollow ZSM-5 or H-ZSM-5 loaded with a catalytic metal or metal oxide (e.g., chromium (Cr), manganese (Mn), iron (Fe), cobalt (Co), nickel (Ni), copper (Cu), zinc (Zn), molybdenum (Mo), or gallium (Ga) and oxides thereof). The metal containing zeolite material can be heated in the presence of a quaternary ammonium cation (e.g., tetramethylammonium, tetraethylammonium, hexadecyltrimethylammonium, dibenzyldimethylammonium, benzyltriethylammonium, and cetyltrimethylammonium) to improve the catalytic properties. It has also been surprisingly found that adding CO₂ to the reactant feed enhances aromatic hydrocarbon yield by lowering the formation of undesired side products, enhancing the formation of olefinic intermediates, which further enhances the yields of aromatic hydrocarbon compounds such as those constituting BTX. Without wishing to be bound by theory, it is believed that CO₂ acts as a soft oxidant and hydrogen scavenger that inhibits side reactions and removes coking from the catalyst surface, which further improves the yield of benzene and other aromatic hydrocarbons. Notably, the catalysts of the present invention provide increased yields and selectivity of benzene, toluene, and xylenes where the combined selectivity of one or more of benzene, toluene, and xylenes is at least 60%, preferably 60% to 90%, or more preferably 80 to 90%.

In one aspect of the present invention, there is disclosed a method for producing aromatic hydrocarbons from a C₃ alcohol, the method including contacting a reactant feed that includes a C₃ alcohol with or without carbon dioxide (CO₂) with a metal doped zeolite catalyst under reaction conditions sufficient to produce an aromatic hydrocarbon containing product stream. The aromatic hydrocarbon containing product stream can include one or more of benzene, toluene, and xylenes, where the combined selectivity of one or more of benzene, toluene, and xylenes is at least 60%, preferably 60% to 90%, or more preferably 80 to 90% at a reaction temperature of at least 450° C., preferably 500° C. to 700° C., or most preferably 550° C. to 650° C. In another aspect the selectivity of benzene is at least 30%, preferably 30% to 50%, the selectivity of toluene is at least 25%, preferably 25% to 40%, and the selectivity of xylenes is at least 5%, preferably 5 to 10%. In a particular aspect, the ratio of C₃ alcohol to CO₂ used in the method is 0≤5 or 0.1≤5, and in one instance the C₃ alcohol is isopropanol and the ratio of CO₂ to isopropanol is 0.1≤5. The isopropanol may also be a mixture of isopropanol and propanol. The zeolite catalyst used in the method can be ZSM-5, H-ZSM-5, a hollow ZSM-5, or a hollow H-ZSM-5 loaded with a catalytic metal, or metal oxide thereof, in an amount of 0.1 wt. % to 20 wt. %, preferably 0.1 wt. % to 10 wt. %, or more preferably 3 wt. % to 7 wt. %. The catalytic metal or metal oxide is a Column 1, 2 and 6-14 (Groups IA, IIA, VIB, VIIB, VIII, VIIIB, IB, IIB, IIIA, IVA, or VA) metal or metal oxide of the Periodic Table, for example sodium (Na), magnesium (Mg), lantheum (La), Ytterbium (Y), vanadium (V), niobium (Nb), molybdenum (Mo), chromium (Cr), manganese (Mn), iron (Fe), cobalt (Co), nickel (Ni), copper (Cu), zinc (Zn), gallium (Ga), indium (In), tin (Sn), antimony (Sb), bismuth (Bi), tellurium (Te), and in a specific embodiment the metal is Ga or Mo. The reaction conditions used in the method can further include a pressure of 0.5 to 5 bar, or 1 to 3 bar, and a weight hourly space velocity (WHSV) of 0.5 h⁻¹ to 20 h⁻¹, 1 h⁻¹ to 10 h⁻¹, or 3 h⁻¹ to 7 h⁻¹. The reactants in the reactant feed can be in the gas phase and the reactant feed can further include a carrier gas. The carrier gas can be an inert gas (e.g., helium or argon) nitrogen, or CO₂. In some aspects, the reactant feed consists essentially of or consists of the C₃ alcohol and CO₂. In some instances, the reactant feed does not include any other alcohol other than a C₃ alcohol. In still some aspects, the reactant feed does not include propylene or propylene. In another aspect, the reactant feed can include from 1 wt. % up to 5 wt. % propanol.

In another embodiment, where the aromatic hydrocarbon containing product stream includes benzene, toluene, and xylene, the combined selectivity of benzene, toluene, and xylenes is at least 30%, 30% to 90%, or preferably 80% to 90%. The reaction conditions of the method include a temperature of at least 200° C., 200° C. to 700° C., 400° C. to 700° C., 500° C. to 700° C., or 550° C. to 650° C., a pressure of 0.5 to 5 bar, or 1 to 3 bar, and a weight hourly space velocity (WHSV) of 0.5 h⁻¹ to 20 h⁻¹, 1 h⁻¹ to 10 h⁻¹, or 3 h⁻¹ to 7 h⁻¹. The zeolite catalyst can be a regular zeolite or a hollow zeolite catalyst (e.g., MFI, FAU, ITH BEA, MOR, LTA, MWW, CHA, MRE, MFE, or a VFI zeolite catalyst), preferably an MFI zeolite catalyst or a hollow MFI zeolite catalyst. The zeolite catalyst used in the method can also be ZSM-5 or H-ZSM-5, wherein the zeolite catalyst is loaded with a catalytic metal or metal oxide. The catalytic metal or metal oxide is a Column 1, 2 and 6-14 (Groups IA, IIA, VIB, VIIB, VIII, VIIIB, IB, IIB, IIIA, IVA, or VA) metal or metal oxide of the Periodic Table, for example Cr, Mn, Fe, Co, Ni, Cu, Zn, Ga, tin (Sn), antimony (Sb), tellurium (Te), and in a specific embodiment the metal is Ga. The zeolite catalyst can include up to 10 wt. % of the metal or metal oxide, preferably 0.1 wt. % to 20 wt. %, more preferably 0.1 wt. % to 10 wt. %, or most preferably 3 wt. % to 7 wt. % or about 5 wt. %. In another aspect, the zeolite catalyst has a Si/Al ratio of less than 100, preferably 5 to 75, more preferably 10 to 60, or most preferably 20 to 55. Without limiting the method to the production aromatic hydrocarbons alone, the method can further include collecting or storing the aromatic hydrocarbon containing product stream and using the produced aromatic hydrocarbon containing product stream to produce a petrochemical or a polymer.

Also disclosed is a system for producing aromatic hydrocarbons using the methods described herein, the system including an inlet for a reactant feed that includes a C₃ alcohol with or without carbon dioxide (CO₂); a reaction zone that is configured to be in fluid communication with the inlet, wherein the reaction zone can include the reactant feed and a metal loaded zeolite catalyst; and an outlet configured to be in fluid communication with the reaction zone to remove an aromatic hydrocarbon containing product stream. The system can further include a collection device that is capable of collecting the aromatic hydrocarbon containing product stream and the reaction zone can be a continuous flow reactor selected from a fixed-bed reactor, a fluidized reactor, or a moving bed reactor. The aromatic hydrocarbon containing product stream can include one or more of benzene, toluene, xylene.

In the context of the present invention there are 50 embodiments described. Embodiment 1 is a method for producing aromatic hydrocarbons from a C₃ alcohol, the method comprising contacting a reactant feed comprising a C₃ alcohol with a catalytic metal-containing zeolite catalyst under reaction conditions sufficient to produce an aromatic hydrocarbon containing product stream. Embodiment 2 is the method of embodiment 1, wherein the reactant feed comprises carbon dioxide (CO₂). Embodiment 3 is the method of any one of embodiments 1 to 2, wherein the aromatic hydrocarbon containing product stream comprises benzene, toluene, and xylenes, and wherein the combined selectivity of benzene, toluene, and xylenes is at least 60%, preferably 60% to 90%, or more preferably 80 to 90% at a reaction temperature of at least 450° C., preferably 500° C. to 700° C., or most preferably 550° C. to 650° C. Embodiment 4 is the method of embodiment 3, wherein the selectivity of benzene is at least 30%, preferably 30% to 50% and the selectivity of toluene is at least 25%, preferably 25% to 40%. Embodiment 5 is the method of embodiment 3, wherein the selectivity of xylenes is at least 5%, preferably 5 to 10%. Embodiment 6 is the method of embodiment 3, wherein the C₃ alcohol is isopropanol and the ratio of CO₂ to isopropanol is 0≤5, preferably 0.1≤5. Embodiment 7 is the method of any one of embodiments 1 to 6, wherein the catalytic metal-containing zeolite comprises up to 10 wt. % of the metal or metal oxide, preferably 0.1 wt. % to 20 wt. %, more preferably 0.1 wt. % to 10 wt. %, or most preferably 3 wt. % to 7 wt. % or about 5 wt. %. oxide thereof, in an amount of 0.1 wt. % to 20 wt. %, preferably 0.1 wt. % to 10 wt. %, or more preferably 3 wt. % to 7 wt. %. Embodiment 8 is the method of embodiment 7, wherein the catalytic metal or metal oxide is a group IA, IIA, VIB, VIIB, VIII, VIIIB, IB, IIB, IIIA, IVA, VA metal or metal oxide. Embodiment 9 is the method of embodiment 8, wherein the catalytic metal is gallium (Ga), nickel (Ni), copper (Cu), iron (Fe), cobalt (Co), chromium (Cr), zinc (Zn) or oxides thereof, or combinations thereof. Embodiment 10 is the method of embodiment 9, wherein the metal or metal oxide comprises Ga. Embodiment 11 is the method of any one of embodiments 1 to 10, wherein the zeolite is ZSM-5, a H-ZSM-5 or a hollow ZSM-5, a hollow H-ZMS-5. Embodiment 12 is the method of any one of embodiments 3 to 11, wherein the reaction conditions further include a pressure of 0.5 to 5 bar, or 1 to 3 bar, and a weight hourly space velocity (WHSV) of 0.5 h⁻¹ to 20 h⁻¹, 1 h⁻¹ to 10 h⁻¹, or 3 h⁻¹ to 7 h⁻¹. Embodiment 13 is the method of any one of embodiments 1 to 12, wherein the C₃ alcohol is isopropanol or a mixture of isopropanol and propanol. Embodiment 14 is the method of any one of embodiments 1 to 13, wherein the ratio of C₃ alcohol to CO₂ is 0≤5. Embodiment 15 is the method of any one of embodiments 1 to 14, wherein the reactants in the reactant feed are in the gas phase. Embodiment 16 is the method of embodiment 15, wherein the reactant feed further comprises a carrier gas. Embodiment 17 is the method of embodiment 16, wherein the carrier gas is helium, argon, nitrogen, or carbon dioxide. Embodiment 18 is the method of any one of embodiments 1 to 17, wherein the reactant feed consists essentially of or consists of the C₃ alcohol and CO₂. Embodiment 19 is the method of any one of embodiments 1 to 18, wherein the reactant feed does not include any other alcohol other than a C₃ alcohol. Embodiment 20 is the method of any one of embodiments 1 to 19, wherein the reactant feed does not include propylene. Embodiment 21 is the method of any one of embodiments 1 to 20, wherein the aromatic hydrocarbon containing product stream comprises benzene, toluene, and xylenes.

Embodiment 22 is the method of embodiment 21, wherein the combined selectivity of benzene, toluene, and xylenes is at least 30%, 30% to 90%, or preferably 80% to 90%. Embodiment 23 is the method of any one of embodiments 1 to 21, wherein the reaction conditions include a temperature of at least 200° C., 200° C. to 700° C., 400° C. to 700° C., 500° C. to 700° C., or 550° C. to 650° C. Embodiment 24 is the method of embodiment 23, wherein the reaction conditions further include a pressure of 0.5 to 5 bar, or 1 to 3 bar, and a weight hourly space velocity (WHSV) of 0.5 h⁻¹ to 20 h⁻¹, 1 h⁻¹ to 10 h⁻¹, or 3 h⁻¹ to 7 h⁻¹. Embodiment 25 is the method of any one of embodiments 1 to 24, wherein the zeolite catalyst is a MFI, FAU, ITH BEA, MOR, LTA, MWW, CHA, MRE, MFE, or a VFI zeolite catalyst, preferably a MFI zeolite catalyst, or a hollow MFI zeolite catalyst. Embodiment 26 is the method of embodiment 25, wherein the MFI zeolite catalyst is ZSM-5, H-ZSM-5, a hollow ZSM-5, or a hollow H-ZSM-5. Embodiment 27 is the method of any one of embodiments 1 to 26, wherein the zeolite catalyst has a Si/Al ratio of less than 100, preferably 5 to 75, more preferably 10 to 60, or most preferably 20 to 55. Embodiment 28 is the method of any one of embodiments 1 to 27, further comprising collecting or storing the aromatic hydrocarbon containing product stream. Embodiment 29 is the method of any one of embodiments 1 to 28, further comprising using the produced aromatic hydrocarbon containing product stream to produce a petrochemical or a polymer.

Embodiment 30 is a system for producing aromatic hydrocarbons, the system comprising: an inlet for a reactant feed comprising a C₃ alcohol and, optionally, carbon dioxide (CO₂); a reaction zone that is configured to be in fluid communication with the inlet, wherein the reaction zone comprises the reactant feed and a catalytic metal-containing zeolite catalyst; and an outlet configured to be in fluid communication with the reaction zone to remove an aromatic hydrocarbon containing product stream. Embodiment 31 is the system of embodiment 30, further comprising a collection device that is capable of collecting the aromatic hydrocarbon containing product stream. Embodiment 32 is the system of any one of embodiments 30 to 31, wherein the reaction zone is a continuous flow reactor selected from a fixed-bed reactor, a fluidized reactor, or a moving bed reactor. Embodiment 33 is the system of any one of embodiments 30 to 32, wherein the C₃ alcohol is isopropanol. Embodiment 34 is the system of any one of embodiments 30 to 33, wherein the ratio of C₃ alcohol to CO₂ is 0≤5. Embodiment 35 is the system of any one of embodiments 30 to 34, wherein the reactants in the reactant feed are in the gas phase. Embodiment 36 is the system of embodiment 35, wherein the reactant feed further comprises a carrier gas. Embodiment 37 is the system of embodiment 36, wherein the carrier gas is helium, argon, nitrogen, or carbon dioxide. Embodiment 38 is the system of any one of embodiments 30 to 37, wherein the reactant feed consists essentially of or consists of the C₃ alcohol and CO₂. Embodiment 39 is the system of any one of embodiments 30 to 38, wherein the reactant feed does not include any other alcohol other than a C₃ alcohol. Embodiment 40 is the system of any one of embodiments 30 to 39, wherein the reactant feed does not include propylene. Embodiment 41 is the system of any one of embodiments 30 to 40, wherein the aromatic hydrocarbon containing product stream comprises benzene, toluene, and xylenes. Embodiment 42 is the system of any one of embodiments 30 to 41, wherein the temperature of the reaction zone is at least 200° C., 200° C. to 700° C., 400° C. to 700° C., 500° C. to 700° C., or 550° C. to 650° C. Embodiment 43 is the system of embodiment 42, wherein the pressure of the reaction zone is 0.5 to 5 bar, or 1 to 3. Embodiment 44 is the system of any one of embodiments 30 to 43, wherein the zeolite catalyst is a MFI, FAU, ITH BEA, MOR, LTA, MWW, CHA, MRE, MFE, or a VFI zeolite catalyst, preferably an MFI zeolite catalyst. Embodiment 45 is the system of embodiment 44, wherein the MFI zeolite catalyst is ZSM-5, H-ZSM-5, a hollow ZSM-5, or a hollow H-ZSM-5. Embodiment 46 is the system of any one of embodiments 29 to 44, wherein the catalytic metal or metal oxide is a group IA, IIA, VIB, VIIB, VIII, VIIIB, IB, IIB, IIIA, IVA, VA metal or metal oxide. Embodiment 47 is the system of embodiment 46, wherein the metal or metal oxide comprises Cr, Mn, Fe, Co, Ni, Cu, Zn, Mo, or Ga. Embodiment 48 is the system of embodiment 47, wherein the metal or metal oxide comprises Ga or Mo. Embodiment 49 is the system of any one of embodiments 46 to 48, wherein the zeolite catalyst comprises up to 10 wt. % of the metal or metal oxide, preferably 0.1 wt. % to 20 wt. %, more preferably 0.1 wt. % to 10 wt. %, or most preferably 3 wt. % to 7 wt. % or about 5 wt. %. Embodiment 50 is the system of any one of embodiments 30 to 49, wherein the zeolite catalyst has a Si/Al ratio of less than 100, preferably 5 to 75, more preferably 10 to 60, or most preferably 20 to 55.

The following includes definitions of various terms and phrases used throughout this specification.

The term “catalyst” means a substance which alters the rate of a chemical reaction. “Catalytic” means having the properties of a catalyst.

The term “mixed metal oxide” catalyst refers to a catalyst that can include metals substantially as oxides or a mixture of metal oxides and metals in other forms (e.g., reduced metal form).

The term “conversion” means the mole fraction (i.e., percent) of a reactant converted to a product or products.

The term “selectivity” refers to the percent of converted reactant that went to a specified product, for example benzene selectivity is the % of isopropanol that formed benzene.

The term “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising”, “including”, “containing”, or “having” in the claims, or the specification, may mean “one,” but it is also consistent with the meaning of “one or more”, “at least one”, and “one or more than one.”

The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods and systems of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the methods and systems of the present invention are their ability to produce aromatic hydrocarbons from a C₃ alcohol.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 is a diagram showing some of the petrochemicals that can be produced from BTX components.

FIG. 2 is a schematic of an embodiment of a system for producing BTX from alkanes.

FIG. 3 is a diagram showing the product yields for the aromatization reaction of isopropanol using a Ga₂O₃/ZSM-5 catalyst at 400° C. and 600° C.

### DETAILED DESCRIPTION OF THE INVENTION

A discovery has been made, which provide methods and systems for producing aromatic hydrocarbons from C₃ alcohol(s). The methods and systems include contacting a reactant feed comprising a C₃ alcohol with or without carbon dioxide (CO₂) with a metal loaded zeolite catalyst under reaction conditions sufficient to produce an aromatic hydrocarbon containing product stream. The methods and systems provide benzene, toluene, and xylenes (BTX) in improved yields over existing methods where the combined selectivity of one or more of benzene, toluene, and xylenes is at least 60%, preferably 60% to 90%, or more preferably 80 to 90%.

In one embodiment a new route for the production of BTX from a C₃ alcohol, specifically, isopropanol in the presence of CO₂ is presented. Aromatic hydrocarbons are prepared from isopropanol and CO₂ by the following reaction schematic:

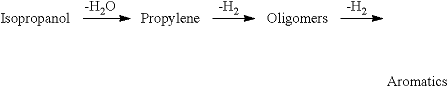

where the conversion of isopropanol to aromatic hydrocarbons involves dehydration of isopropanol to olefin, olefin oligomerization, cyclization, dehydrogenation, and aromatization steps. Both thermodynamics calculations and experimental results show that isopropanol can be converted to aromatic hydrocarbons in the presence of CO₂ at temperatures >200° C. with high conversion. The current method and systems provide increased yields of benzene (more than 50%) at higher temperature. Zeolitic acidity contributes to facile dehydration of isopropanol and it is believed that the use of CO₂ as soft oxidant and hydrogen scavenger that inhibits side reactions and removes coking from the catalyst surface, which further improves the yield of benzene and other aromatic hydrocarbons. Notably, the reaction can be performed at lower temperatures (e.g., 200° C.) as compared to conventional catalysts. In the reaction scheme above, the acidity of the zeolite can promote the formation of alkenes from the alcohol, and the formation of oligomers from the alkenes. Dehydrogenation of the oligomers to aromatic hydrocarbons can be promoted through the zeolite catalyst or through the catalytic metal loaded on the zeolite catalyst. Due to the presence of carbon dioxide, coking of the zeolite catalyst and/or sintering of catalytic metal is inhibited, thereby promoting higher selectivity and yield of aromatic products instead of formation of side products (e.g., cracking products).

These and other non-limiting aspects of the present invention are discussed in further detail in the following sections.

### A. Zeolite Catalyst

The catalysts of the present invention are capable of producing aromatic hydrocarbons from a C₃ alcohol and carbon dioxide (CO₂) in a single pass. In some embodiments, carbon dioxide is not used. The catalysts used in the present invention are typically metal loaded zeolites, or hollow metal loaded zeolites. Zeolites can be an effective substrate, are commercially available, and are well known in the art. Zeolites are typically microporous, aluminosilicate crystalline minerals commonly used as commercial adsorbents and catalysts in the petrochemical industry, for instance in fluid catalytic cracking (FCC) and hydro-isomerization processes. The zeolite catalysts of the current invention can be a MFI, FAU, ITH BEA, MOR, LTA, MWW, CHA, MRE, MFE, or a VFI zeolite catalyst. In a specific embodiment the catalyst is a metal loaded MFI zeolite catalyst, such as a metal loaded ZSM-5 or metal loaded H-ZSM-5 which is ZSM-5 in its protonated form. The metal loaded zeolite catalyst can also be loaded with a promotor element such as a catalytic metal or metal oxide and loading can be accomplished by know processes in the art, including ion exchange, impregnation, etc. Typically, zeolite catalysts used to prepare petrochemicals suffer from coke formation that leads to rapid catalyst deactivation. Encapsulating metal particles inside the hollow structure of the zeolite framework shields these metal particles from sintering which can decrease or prevent catalyst deactivation. One or more of the catalysts of the current embodiments can include a zeolite catalyst that contains metals (e.g., metals in reduced form), metal compounds (e.g., metal oxides) or mixtures thereof (“collectively metals”) of Column 1 or 2 metals, transition metals, post-transition metals, and lanthanides (atomic number 57-71) of the Periodic Table. Non-limiting examples of transition metals and post-transition metals include chromium (Cr), molybdenum (Mo), tungsten (W), manganese (Mn), iron (Fe), ruthenium (Ru), cobalt (Co), rhodium (Rh), nickel (Ni), palladium (Pd), copper (Cu), silver (Ag), zinc (Zn), cadmium (Cd), and gallium (Ga). Specifically, the catalytic metal or metal oxide can be a Group IA, IIA, VIB, VIIB, VIII, VIIIB, IB, IIB, IIIA, IVA, or VA metal or metal oxide, for example sodium (Na), magnesium (Mg), lantheum (La), Ytterbium (Y), vanadium (V), niobium (Nb), molybdenum (Mo), chromium (Cr), manganese (Mn), iron (Fe), cobalt (Co), nickel (Ni), copper (Cu), zinc (Zn), gallium (Ga), indium (In), tin (Sn), antimony (Sb), bismuth (Bi), tellurium (Te), or mixtures thereof. In a particular aspect, the metal or metal oxide comprises Ga or Mo. The zeolite catalyst of the present invention can include up to 20 wt. % of the metal and/or metal oxide, from 0.1 wt. % to 20 wt. %, from 1 wt. % to 10 wt. %, or from 3 wt. % to 7 wt. % and all wt. % there between including 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5 wt. %, 5.1 wt. %, 5.2 wt. %, 5.3 wt. %, 5.4 wt. %, 5.5 wt. %, 5.6 wt. %, 5.7 wt. %, 5.8 wt. %, 5.9 wt. %, 6 wt. %, 6.1 wt. %, 6.2 wt. %, 6.3 wt. %, 6.4 wt. %, 6.5 wt. %, 6.6 wt. %, 6.7 wt. %, 6.8 wt. %, and 6.9 wt. %. In a specific embodiment, the zeolite catalyst includes about 5 wt. % of metal and/or metal oxide. The zeolite catalyst of the present invention can have a Si/Al ratio of less than 100, from 5 to 75, from 10 to 60, or from 20 to 55 and all ratios there between including 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54. The metals used to prepare the zeolite catalyst of the present invention can be provided in varying oxidation states as metallic, oxide, hydrate, or salt forms typically depending on the propensity of each metals stability and/or physical/chemical properties. The metals in the catalyst can also exist in one or more oxidation states. Preferably, the metals or metal oxides used in the preparation of the mixed metal oxide catalyst are provided in stable oxidation states as complexes with monodentate, bidentate, tridentate, or tetradendrate coordinating ligands such as for example iodide, bromide, sulfide, thiocyanate, chloride, nitrate, azide, fluoride, hydroxide, oxalate, water, isothiocyanate, acetonitrile, pyridine, ammonia, ethylenediamine, 2,2′-bipyridine, 1,10-phenanthroline, nitrite, triphenylphosphine, cyanide, carbon monoxide, or mixtures thereof. In a preferred aspect, the metals are impregnated into the zeolite catalysts as aqueous solutions of metal nitrate, metal nitrate hydrates, metal nitrate trihydrates, metal nitrate hexahydrates, and metal nitrate nonahydrates for example gallium nitrate hydrate (Ga(NO₃)₃•H₂O), nickel nitrate hexahydrate (Ni(NO₃)₂•6H₂O), copper nitrate trihydrate Cu(NO₃)₂•3H₂O, iron nitrate nonahydrate (Fe(NO₃)₃•9H₂O), cobalt nitrate hexahydrate (Co(NO₃)₂•6H₂O), chromium nitrate nonahydrate (Cr(NO₃)₃•9H₂O), ammonium heptamolybdate (NH₄)₆—Mo₇O₂₄•4H₂O, and zinc nitrate hexahydrate (Zn(NO₃)₂•6H₂O). A non-limiting example of a commercial source of the above mentioned metals and metal oxides is Sigma Aldrich® (U.S.A).

### B. Methods of Making Zeolite Catalysts

The catalysts of the present invention can be prepared by processes known to those having ordinary skill in the art. By way of example, the catalysts can be prepared by liquid-liquid blending, solid-solid blending, or liquid-solid blending (e.g., any of precipitation, co-precipitation, impregnation, complexation, gelation, crystallization, microemulsion, sol-gel, solvothermal, hydrothermal, sonochemical, or combinations thereof). The mixed metal oxide zeolite catalysts of the present invention can be prepared by impregnation of any of the above mentioned metals, metal oxides, or mixtures thereof, followed by drying and further heating in the presence of a quaternary ammonium salt or amines Non-limiting examples of quaternary ammonium salts include tetrapropylammioum hydroxide (TPAOH), tetraethylammonium hydroxide (TEAOH), tetramethylammonium hydroxide (TMAOH), hexadecyltrimethylammonium hydroxide, dibenzyldimethylammonium hydroxide, benzyltriethylammonium hydroxide, and cetyltrimethylammonium hydroxide or alkyl derivatives thereof. Non-limiting examples of amines include diisopropylamine (DIPA), diisopropylethylamine (DIPEA), morpholine, piperidine, pyrrolidine, diethylamine (DEA), triethylamine (TEA), or alkyl derivatives thereof. Heating the metal loaded zeolite in the presence of a quaternary ammonium salts and/or amines can have profound effects on the resultant crystal morphology (size, shape, dispersion, surface area, distribution), and thus the activity of the zeolite catalyst formed. The pH of the solution can be adjusted to assist in the dissociation of the counter ion (e.g., nitrate, oxalate, chloride, sulfide etc.) from the metal oxide. Without wishing to be bound by theory, it is believed that the heating the metal loaded zeolite in the presence of the hydroxide salt can preferentially dissolve the silica in the zeolite structure. The treated zeolite and/or hollow zeolite can have an increased surface area, increased micropore surface area, an increased surface Si/Al ratio, and an increased amount of strong acid sites, all of which facilitating hydrogen transfer reactions. The quaternary ammonium salts and/or amines can be mixed or suspended with the metal impregnated zeolite catalyst from 2 ml/g zeolite to 6 ml/g zeolite, or from 3 ml/g zeolite to 5 ml/g zeolite and all value there between including about 3.1 ml/g zeolite, about 3.2 ml/g zeolite, about 3.3 ml/g zeolite, about 3.4 ml/g zeolite, about 3.5 ml/g zeolite, about 3.6 ml/g zeolite, about 3.7 ml/g zeolite, about 3.8 ml/g zeolite, about 3.9 ml/g zeolite, about 4 ml/g zeolite, about 4.1 ml/g zeolite, about 4.2 ml/g zeolite, about 4.3 ml/g zeolite, about 4.4 ml/g zeolite, about 4.5 ml/g zeolite, about 4.6 ml/g zeolite, about 4.7 ml/g zeolite, about 4.8 ml/g zeolite, about 4.9 ml/g zeolite, and in a specific embodiment 4.15 ml/g zeolite.

In a non-limiting example of the present invention, catalyst of the present invention can be prepared in a stepwise fashion by first loading the zeolite (ZSM-% with Si/Al ratio of 23, 30, or 50) with an appropriate aqueous metal solution (e.g., Ga, Ni, Cu, Fe, Co, Cr, Mo, or Zn) and then dried. In the second step, the metal loaded zeolite catalyst can be heated in the presence of an aqueous solution of a quaternary ammonium salt (e.g., TPAOH). The precipitate from either step can be collected by standard techniques, such as decanting, filtration, or centrifuging. In a preferred aspect the precipitate formed from the metal loading (e.g., impregnation) is dried at 40° C. to 60° C., specifically 50° C. under air from 8 hours to 12 hours. In another preferred aspect the metal loaded zeolite precipitate is heated with the quaternary ammonium salt under hydrostatic conditions (e.g., autoclave) at a temperature from about 140° C. to about 200° C., preferably 170° C. for a period time ranging from about 12 hours to about 26 hours, preferably 24 hour. The mixture can then be centrifuged at a range from about 3000 rpm to about 7000 rpm, from about 4000 rpm to about 6000 rpm, and preferably about 5000 rpm for anywhere between 10 minutes and 30 minutes, preferably 15 minutes followed by drying overnight at temperature from about 100° C. to about 120° C., preferably 110° C. overnight to obtain a metal loaded zeolite catalyst precursor. Multiple centrifugations can be performed, washing with water in between. In some aspects, the final zeolite catalysts of the present invention are prepared under oxidative conditions (e.g., calcination) and the metals included in the zeolite catalyst are present in higher oxidation states, for example as oxides. The dried templated catalyst precursor can be calcined for 2 to 24 hours, specifically about 12 hours at a temperature of 250 to 800° C., specifically about 525° C. under airflow to obtain an active zeolite catalyst.

The zeolite catalysts of the present invention can be ground into a fine powder, micronized or nanonized to desired mesh particle size distributions, or pressed into pellets, crushed, and sieved to particle size ranges from about 100 μm to about 600 μm, from about 200 μm to about 500 μm, and preferably between 250 m and 425 μm. Without wishing to be bound by theory, it is believed that the catalyst activity depends on the particle size of the metals in the mixed metal oxide zeolite catalyst, which depends mainly on electronic effects, as the electron density at the active sites (on the surface) can vary due to particle size. This effect can be closely related to particle shape and the number of low coordination sites (edges and corners) on the surface as well as the composition of the catalyst.

### C. CO₂/Isopropanol Feed Stream

Isopropanol and carbon dioxide used in the present invention can be obtained from various sources. Isopropanol and carbon dioxide may be purchased in various grades from commercial sources. Alternatively, isopropanol may be prepared by the hydration of propene or by hydrogenating acetone. In some embodiments, the isopropanol includes n-propanol. In one non-limiting instance, the carbon dioxide can be obtained from a waste or recycle gas stream (e.g. from a plant on the same site, like for example from ammonia synthesis) or after recovering the carbon dioxide from a gas stream. A benefit of recycling such carbon dioxide as a starting material in the process of the invention is that it can reduce the amount of carbon dioxide emitted to the atmosphere (e.g., from a chemical production site). The CO₂/isopropanol reactant gas stream ratio for the aromatization reaction can be 0≤5 or 0.1≤5 and the remainder of the reactant gas stream can include another gas or gases provided the gas or gases are inert, such as helium (He), argon (Ar), or nitrogen (N₂), and do not negatively affect the reaction. All possible percentages of isopropanol±CO₂±inert gas are envisioned in the current embodiments as having the described CO₂/isopropanol ratios herein. In another embodiment, the isopropanol can be a mixture of isopropanol and propanol and/or the reactant feed does not include any other alcohol and other than C₃ alcohols. In still another embodiment, the reactant feed does not include propylene or propene. For example, in one instance the reactant feed stream includes 20 vol. % CO₂ and 80 vol. % isopropanol. In another instance, the reactant feed stream includes 20 vol. % He and 80 vol. % isopropanol. Preferably, the reactant mixture is highly pure and substantially devoid of impurities.

### D. Aromatic Hydrocarbon Production System

Conditions sufficient for the aromatization reaction of isopropanol include temperature, time, space velocity, and pressure. The temperature range for the aromatization reaction can be at least 200° C. and ranges from 200° C. to 700° C., from 400° C. to 700° C., from 500° C. to 700° C., and in a specific embodiment from 550° C. to 650° C. and all temperatures in between including 551° C., 552° C., 553° C., 554° C., 555° C., 556° C., 557° C., 558° C., 559° C., 560° C., 561° C., 562° C., 563° C., 564° C., 565° C., 566° C., 567° C., 568° C., 569° C., 570° C., 571° C., 572° C., 573° C., 574° C., 575° C., 576° C., 577° C., 578° C., 579° C., 580° C., 581° C., 582° C., 583° C., 584° C., 585° C., 586° C., 587° C., 588° C., 589° C., 590° C., 591° C., 592° C., 593° C., 594° C., 595° C., 596° C., 597° C., 598° C., 600° C., 601° C., 602° C., 603° C., 604° C., 605° C., 606° C., 607° C., 608° C., 609° C., 610° C., 611° C., 612° C., 613° C., 614° C., 615° C., 616° C., 617° C., 618° C., 619° C., 620° C., 621° C., 622° C., 623° C., 624° C., 625° C., 626° C., 627° C., 628° C., 629° C., 630° C., 631° C., 632° C., 633° C., 634° C., 635° C., 636° C., 637° C., 638° C., 639° C., 640° C., 641° C., 642° C., 643° C., 644° C., 645° C., 646° C., 647° C., 648° C., and 649° C. The gas hourly space velocity (GHSV) for the aromatization reaction can range from about 0.5 h⁻¹ to about 20 h⁻¹, from about 1 h⁻¹ to about 10 h⁻¹, and in a specific embodiment from about 3 h⁻¹ to about 7 h⁻¹ and all GHSV in between including 3.1 h⁻¹, 3.2 h⁻¹, 3.3 h⁻¹, 3.4 h⁻¹, 3.5 h⁻¹, 3.5 h⁻¹, 3.6 h⁻¹, 3.7, h⁻¹, 3.8 h⁻¹, 3.9 h⁻¹, 4 h⁻¹, 4.1 h⁻¹, 4.2 h⁻¹, 4.3 h⁻¹, 4.4 h⁻¹, 4.5 h⁻¹, 4.5 h⁻¹, 4.6 h⁻¹, 4.7 h⁻¹, 4.8 h⁻¹, 4.9 h⁻¹, 5 h⁻¹, 5.1 h⁻¹, 5.2 h⁻¹, 5.3 h⁻¹, 5.4 h⁻¹, 5.5 h⁻¹, 5.5 h⁻¹, 5.6 h⁻¹, 5.7, h⁻¹, 5.8 h⁻¹, 5.9 h⁻¹, 6 h⁻¹, 6.1 h⁻¹, 6.2 h⁻¹, 6.3 h⁻¹, 6.4 h⁻¹, 6.5 h⁻¹, 6.5 h⁻¹, 6.6 h⁻¹, 6.7, h⁻¹, 6.8 h⁻¹, and 6.9 h⁻¹. The average pressure for the aromatization reaction can range from about 0.5 bar to about 5 bar, and in a specific embodiment from about 1 bar to about 3 bar and all pressures in between including 1.1 bar, 1.2 bar, 1.3 bar, 1.4 bar, 1.5 bar, 1.6 bar, 1.7 bar, 1.8 bar, 1.9 bar, 2 bar, 2.1 bar, 2.2 bar, 2.3 bar, 2.4 bar, 2.5 bar, 2.6 bar, 2.7 bar, 2.8 bar, and 2.9 bar, or more. The upper limit on pressure can be determined by the reactor used. The conditions for the aromatization reaction of isopropanol can be varied based on the type of the reactor.

In another aspect, the reaction can be carried out over the zeolite catalyst of the current invention having the particular aromatic hydrocarbon selectivity and conversion. In one embodiment the aromatic hydrocarbon containing product stream can include one or more of benzene, toluene, and xylenes (BTX), where the combined selectivity of one or more of benzene, toluene, and xylenes can be at least 60%, preferably 60% to 90%, or more preferably 80 to 90% and all selectivity in between including 61, 62, 63, 64, 65, 66, 67, 68, 69, 70, 71, 72, 73, 74, 75, 76, 77, 78, 79, 80, 81, 82, 83, 84, 85, 86, 87, 88, and 89%. In another embodiment the selectivity of benzene is at least 30%, preferably 30% to 50% and all selectivity in between including 31, 32, 33, 34, 35, 36, 37, 38, 39, 40, 41, 42, 43, 44, 45, 46, 47, 48, and 49, and the selectivity of toluene is at least 25%, preferably 25% to 40% and all selectivity in between including 26, 27, 28, 29, 30, 31, 32, 33, 34, 35, 36, 37, 38, and 39, and the selectivity of xylenes is at least 5%, preferably 5 to 10% and all selectivity in between including 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, and 9.9%. Xylenes exists as several dimethylbenzene constitutional isomers including ortho-xylene (1,2-dimethylbenzene), meta-xylene (1,3-dimethylbenzene), and para-xylene (1,4-dimethylbenzene). Other alkylbenzenes formed during the aromatization reaction include one or more of ethylbenzene (EB) and trimethylbenzenes (TMB), tert-butylbenzene, isobutylbenzene, sec-butylbenzene, n-butylbenzene, 1-methyl-2-ethylbenzene, 1-methyl-3-ethylbenzene, 1-methyl-4-isopropylbenzene, 1-methyl-2-n-propylbenzene, 1-methyl-4-n-propylbenzene, 1-methyl-3-n-propylbenzene, 1,3-dimethyl-5-ethylbenzene, 1,4-dimethyl-2-ethylbenzene 1,2-dimethyl-4-ethylbenzene, 1,3-dimethyl-2-ethylbenzene, 1,2,4,5,-tetramethylbenzene, 1,2-diethylbenzene, 2-methylbutylbenzene, 1,2-diethylbenzene, 2-methylbutylbenzene, tert-1-butyl-2-methylbenzene, tert-1-butyl-4-ethylbenzene, 1,2,4-triethylbenzene, 1,3,5-triethylbenzene, n-hexylbenzene. TMB exists as a mixture of 1,2,3-trimethylbenzene (hemellitene), 1,2,4-trimethylbenzene (pseudocumene), and 1,3,5-trimethylbenzene (mesitylene). The composition of benzene, toluene, ethylbenzene, and xylenes is referred to as BTEX.

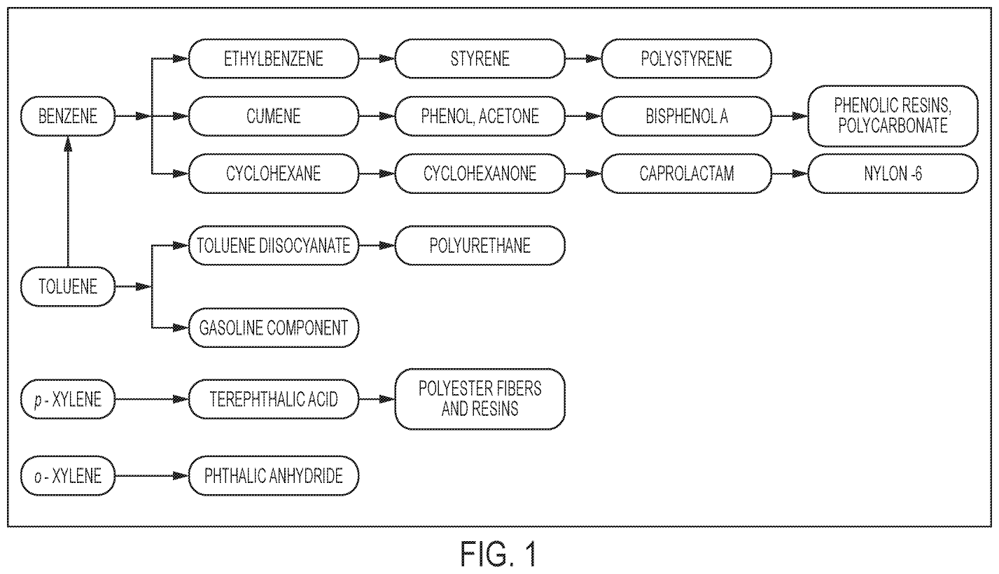

The zeolite catalyst may be used for prolonged periods of time without changing or re-supplying new catalyst or preforming catalyst regeneration. This is due to the stability or slower deactivation of the catalysts of the present invention. Therefore, in one aspect the reaction can be performed wherein one pass aromatic selectivity is 10 to 100%, preferably, 40 to 90%, or more preferably from 60 to 95% after 3 hours to 10 hours on the stream. In another aspect, the one pass alcohol conversion is 10% to 100% after 3 hours to 10 hours on the stream and the catalysts of the present invention remain 60 to 90% active, preferably 70 to 85% active, after 10 hours of time on the stream. The method can further include collecting or storing the produced aromatic hydrocarbons along with using the produced aromatic hydrocarbons, after separation, as a feed source for petrochemical products or a polymer. By way of example only, FIG. 1 provides non-limiting uses of the constituents of BTX produced from the method and systems of the present invention. In some aspects, before use, the zeolite catalysts of the present invention are treated under oxidative conditions (e.g., calcination) and the metals included in the zeolite catalyst are present in higher oxidation states, for example as oxides. The zeolite catalyst can be calcined for 1 to 24 hours, specifically about 12 hours at a temperature of 250 to 800° C., specifically about 525° C. under airflow to obtain the desired oxidized zeolite catalyst.

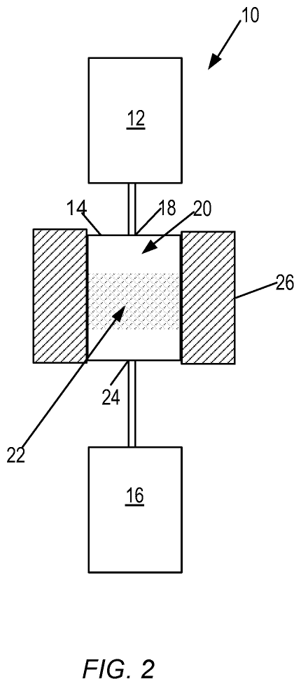

Referring to FIG. 2, system 10 is illustrated, which can be used to convert a C₃ alcohol and carbon dioxide to aromatic hydrocarbons using the zeolite catalysts of the present invention. The system 10 can include a feed source 12, a reactor 14, and a collection device 16. The feed source 12 can be configured to be in fluid or gas communication with the reactor 14 via an inlet 18 on the reactor. The feed can comprise any of the reactants disclosed throughout the current disclosure including but not limited to mixtures of a C₃ alcohol and gas such as isopropanol and carbon dioxide or helium. As explained above, the feed source 12 can be configured such that it regulates the amount of reactant feed entering the reactor 14. As shown, the C₃ alcohol and carbon dioxide feed source 12 is one unit feeding into one inlet 18, however, it should be understood that the number of inlets and/or separate feed sources can be adjusted to reactor sizes and/or configurations. The reactor 14 can include a reaction zone 20 having the metal loaded zeolite catalyst 22 of the present invention. The reactor can include various automated and/or manual controllers, valves, heat exchangers, gauges, etc. necessary for the operation of the reactor. The reactor can be have the necessary insulation and/or heat exchangers to heat or cool the reactor as necessary. The amounts of the C₃ alcohol and carbon dioxide feed and the metal loaded zeolite catalyst 22 used can be modified as desired to achieve a given amount of product produced by the system 10. Non-limiting examples of continuous flow reactors that can be used include fixed-bed reactors, fluidized reactors, bubbling bed reactors, slurry reactors, rotating kiln reactors, moving bed reactors or any combinations thereof when two or more reactors are used.

In preferred aspects, reactor 14 is a continuous flow fixed-bed reactor. The reactor 14 can include an outlet 24 configured to be in fluid communication with the reaction zone and configured to remove a first hydrocarbon product stream comprising aromatic hydrocarbons from the reaction zone 20. Reaction zone 20 can further include the reactant feed and the first product stream. The products produced can include benzene, toluene, and xylene (BTX). In some aspects, the catalyst can be included in the product stream. The collection device 16 can be in fluid communication with the reactor 14 via the outlet 24. Both the inlet 18 and the outlet 24 can be opened and closed as desired. The collection device 16 can be configured to store, further process, or transfer desired reaction products (e.g., benzene, toluene, and xylenes) for other uses. In a non-limiting example, collection device can be a separation unit or a series of separation units that are capable of separating the liquid components from the gaseous components from the product stream. The resulting aromatic hydrocarbons can be sold, stored or used in other processing units as a feed source. Still further, the system 10 can also include a heating/cooling source 26. The heating/cooling source 26 can be configured to heat or cool the reaction zone 20 to a temperature sufficient (e.g., 400 or 600° C.) to convert a C₃ alcohol in the reactant feed to aromatic hydrocarbons. Non-limiting examples of a heating/cooling source 20 can be a temperature controlled furnace or an external, electrical heating block, heating coils, or a heat exchanger.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Catalyst Preparation

### Example 1A

### Synthesis of Metal/ZSM-5

Zeolites (ZSM-% with Si/Al ratio of 23, 30 and 50) were used as a catalyst support. The support was impregnated with aqueous solutions of Ga, Ni, Cu, Fe, Co, Cr, Zn (Ga(NO₃)₃•H₂O, Ni(NO₃)₂•6H₂O, Cu(NO₃)₂•3H₂O, Fe(NO₃)₃•9H₂O, Co(NO₃)₂•6H₂O, Cr(NO₃)₃•9H₂O, Zn(NO₃)₂•6H₂O), or (NH₄)₆—Mo₇O₂₄•4H₂O. The suspension was dried at 50° C. under air overnight then calcined at 550° C. in air for 12 h.

### Example 1B

### Synthesis of Hollow Metal/ZSM-5

Zeolites (ZSM-% with Si/Al ratio of 23, 30 and 50) were used as a catalyst support. The support was impregnated with aqueous solutions of Ga, Ni, Cu, Fe, Co, Cr, Zn Zeolites (ZSM-% with Si/Al ratio of 23, 30 and 50) were used as a catalyst support. The support was impregnated with aqueous solutions of Ga, Ni, Cu, Fe, Co, Cr, Zn (Ga(NO₃)₃•H₂O, Ni(NO₃)₂•6H₂O, Cu(NO₃)₂•3H₂O, Fe(NO₃)₃•9H₂O, Co(NO₃)₂•6H₂O, Cr(NO₃)₃•9H₂O, Zn(NO₃)₂•6H₂O), or (NH₄)₆—Mo₇O₂₄•4H₂O. The suspension was dried at 50° C. under air overnight then calcined at 550° C. in air for 12 h. (Ga(NO₃)₃•H₂O, Ni(NO₃)₂•6H₂O, Cu(NO₃)₂•3H₂O, Fe(NO₃)₃•9H₂O, Co(NO₃)₂•6H₂O, Cr(NO₃)₃•9H₂O, Zn(NO₃)₂•6H₂O), or ammonium heptamolybdate (NH₄)₆—Mo₇O₂₄•4H₂O. The suspension was dried at 50° C. under air overnight. The impregnated zeolite was then suspended with tetrapropylammonium hydroxide (4.15 ml/g zeolite) and water (3.33 ml/g zeolite). The mixture was transferred into Teflon-lined autoclave and heated at 170° C. under static conditions for 24 h. The solid was recovery by centrifugation and washed with water, this operation was repeated 3 times. The resulting solid was dried overnight at 110° C. and then calcined for 12 h at 525° C. in air.

### Example 2

### Catalyst Testing

General Procedure. Catalyst testing was performed in a fixed bed reactor with 0.5 cm in diameter and 25 cm in length. The effluent of the reactor is connected to Agilent gas chromatography (GC) 7890 A for online gas analysis. The catalyst was pressed into pellets then crushed and sieved between 250-425 μm. A catalyst sieved fraction (0.25 ml) was placed on top of porous plate inside the reactor. Prior to the reaction test, the catalyst was calcined at 550° C. for 30 min in air. For A mixture of isopropanol/He or isopropanol/CO₂ with weight hourly space velocity (WHSV) of about 1.74 h⁻¹ was introduced into the reactor at atmospheric pressure and different reaction temperature (e.g., 400° C., 450° C., and 600° C.). Isopropanol conversion as well as products selectivity and yield were calculated as follows:

$${X}_{Isopro} = \frac{{\sum }_{1}^{i} {nC}_{i}}{{[ Isopro ]}_{in}}$$

$${S}_{Ci} = \frac{[ {C}_{i} ]}{{\sum }_{1}^{i} {nC}_{i}}$$

$${Y}_{Ci} = {X}_{Isopro} \times {S}_{Ci}$$

n=number of carbon atoms.

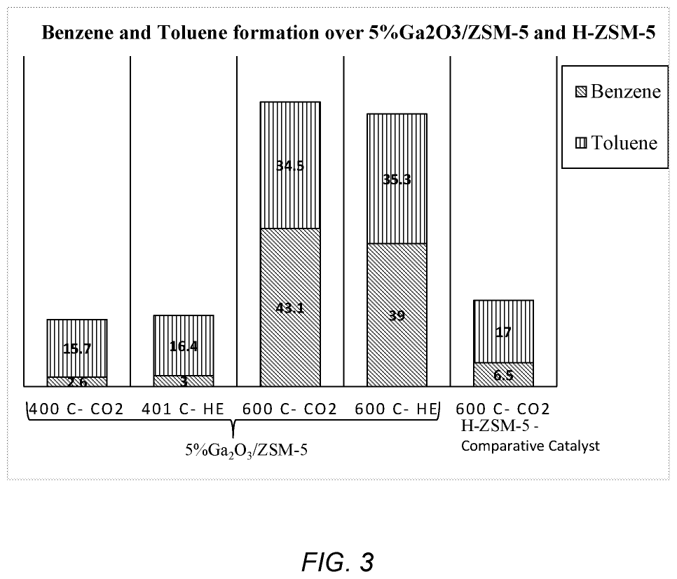

Ga₂O₃/ZSM-5 catalyst. The Ga₂O₃/ZSM-5 catalyst as prepared in Example 1 was tested as described above, and the results are tabulated in Table 1. FIG. 3 shows the isopropanol aromatization over a 5% Ga₂O₃/ZSM-5 catalyst. The results showed that high temperature yields higher aromatics compare to lower temperature. For example under He flow, the sum (E) of all aromatics at 400° C. was 46.2% while it was 84.4% at 600° C. When CO₂ was used as co-reactant with isopropanol, the ΣAromatics was 46% and 86.2% at 400 and 600° C., respectively. Using CO₂ enhances the product yield by controlling the formation of undesired side products. Oxidative dehydrogenation mediated by CO₂ as soft oxidant enhances the formation of olefinic intermediates, which further enhances the yields of aromatic compounds.

Dehydration, dehydrogenation and aromatization of isopropanol performed over Ga-ZSM-5 catalysts showed very high formation of C₃ and C₄ along with benzene. The competitive formation of C₃ and C₄ olefins was taken into consideration, which shows the major product as C₄ olefins at low temperature. The C₄ fraction containing butane(s) and butadiene are up to 40% of the product distribution among C₁-C₅ with 30% is C₃ olefin and the rest 30% is C₂. The products can be tuned based on the morphology of the catalysts and reaction conditions. Formation of benzene by oligomerization of C₃ is highly favored at high temperatures above 600° C. where benzene is the major product among all products constituting up to 60% of the products along with toluene.

Dehydration, dehydrogenation and aromatization of isopropanol performed over Mo-ZSM-5 catalysts showed very high formation of C₃ and C₄ along with benzene. Formation of toluene by oligomerization of C₃ is highly favored at high temperatures above 600° C. where toluene is the major product among all products.

Comparative H-ZSM-5 catalyst. A comparative H-ZSM-5 catalyst that did not include metal loading was tested using the procedure outlined above. The results are tabulated in Table 3. Comparing the catalyst of the present invention to a non-metal loaded zeolite catalyst, at 600° C. using a CO₂ reactant stream, the amount of benzene produced was increased from 6.2% to 43.12%, the amount of toluene was increased from 17% to 34.52 and the ΣAromatics increased from 35.3% to 86.2%.

TABLE 1

| Yield, | | | | | | | | | | | | | | | | | | | | |
|---|---|---|---|---|---|---|---|---|---|---|---|---|---|---|---|---|---|---|---|---|
| % C | Temp | | | C₂ | | C₃ | | | | | | | | | | p&m- | | | | |
| based | (° C.) | Gas | C₁ | olefin | C₂ | olefin | C₃ | C₄ | C₅ | C₆₊ | Bz | C₇ | Tol | C₈ | EB | X | o-X | TMB | C₉+ | ΣAy |
| 5 wt. %, | 450 | He | .04 | 2.38 | .14 | 5.64 | 8.08 | 23.15 | 8.27 | 9.71 | 2.07 | | 16.41 | | 1.94 | 8.37 | 6.95 | 5.31 | 0.57 | 42.5 |
| Ga₂O₃/ | (1 h) | CO₂ | .03 | 2.06 | .13 | 5.11 | 7.34 | 22.80 | 8.76 | 6.09 | 2.62 | | 15.72 | | 2.18 | 15.20 | 4.15 | 6.15 | 1.66 | 47.7 |
| ZSM-5 | 600 | He | .95 | 5.99 | .71 | 2.66 | 2.09 | .31 | 0 | 2.84 | 38.99 | 0 | 35.3 | 0 | .37 | 4.07 | 2.57 | 3.14 | 0 | 84.4 |
| | (3 H) | CO₂ | .91 | 5.6 | .58 | 1.62 | .92 | .17 | 0 | 0 | 43.12 | 0 | 34.52 | 0 | .29 | 5.28 | 2.53 | .51 | 3.96 | 86.2 |

EB = ethylbenzene,
TMB = trimethylbenzene,
X = xylene

TABLE 2

| Yield, | | | | | | | | | | | | | | | | | | |
|---|---|---|---|---|---|---|---|---|---|---|---|---|---|---|---|---|---|---|
| % C | Temp | | | C₂ | | C₃ | | | | | | | | p&m- | | | | |
| based | (° C.) | Gas | C₁ | olefin | C₂ | olefin | C₃ | C₄ | C₅ | C₆₊ | Bz | Tol | EB | X | o-X | TMB | C₉+ | ΣAy |
| 1 wt. % | 450 | He | 0.1 | 5.33 | 0.4 | 10.3 | 10.7 | 19.9 | 5.7 | 12.7 | 4.4 | 16.9 | 1.1 | 14.6 | 0.2 | 3.6 | 0.9 | 41.6 |
| MoOₓ/ | (1 h) | CO₂ | .08 | 5.07 | 0.4 | 9.97 | 10.3 | 19.5 | 5.6 | 9.5 | 4.5 | 17.2 | 1.1 | 15.0 | 4.3 | 3.8 | 1.2 | 46.2 |
| hollow | 600 | He | .82 | 20.9 | 1.1 | 19.4 | 4.0 | 5.6 | 0.9 | 3.8 | 11.5 | 22.7 | 0.4 | 7.1 | 2.4 | 1.0 | 1.2 | 47.1 |
| H-ZSM-5 | (3 H) | CO₂ | .82 | 19.0 | 1.0 | 14.7 | 3.2 | 3.9 | 0.8 | 4.4 | 14.6 | 27.6 | 0.4 | 8.5 | 2.9 | 1.1 | 1.0 | 56.0 |

EB = ethylbenzene,
TMB = trimethylbenzene,
X = xylene

TABLE 3

| % C | Temp | | | C₂ | | C₃ | | | | | | | | | | p&m- | | | | |
|---|---|---|---|---|---|---|---|---|---|---|---|---|---|---|---|---|---|---|---|---|
| based | (° C.) | Gas | C₁ | olefin | C₂ | olefin | C₃ | C₄ | C₅ | C₆ | Bz | C₇ | To! | C₈ | EB | X | o-X | TMB | C₉+ | ΣAy |
| 5 wt. % | 600 | CO2 | 1.2 | 12.9 | 6.5 | 16.4 | 13.5 | 9.7 | 1.7 | 1.9 | 6.5 | .3 | 17 | 0 | .5 | 7.7 | 2.4 | 1.2 | .6 | 35.3 |
| H-ZSM-5 | | | | | | | | | | | | | | | | | | | | |

1. A method for producing aromatic hydrocarbons from a C₃ alcohol, the method comprising contacting a reactant feed comprising a C₃ alcohol with a catalytic metal-containing zeolite catalyst under reaction conditions sufficient to produce an aromatic hydrocarbon containing product stream, wherein the aromatic hydrocarbon containing product stream comprises benzene, toluene, and xylenes, and wherein the combined selectivity of benzene, toluene, and xylenes is at least 60% at a reaction temperature of from 555° C. to 650° C.

2. The method of claim 1, wherein the reactant feed comprises carbon dioxide (CO₂).

3. The method of claim 1, wherein the aromatic hydrocarbon containing product stream comprises benzene, toluene, and xylenes, and wherein the combined selectivity of benzene, toluene, and xylenes is 60% to 90%.

4. The method of claim 1, wherein the selectivity of benzene is at least 30%, or the selectivity of toluene is at least 25%, or the selectivity of xylenes is at least 5%.

5. The method of claim 4, wherein the C₃ alcohol is isopropanol and the ratio of CO₂ to isopropanol is 0≤5.

6. The method of claim 1, wherein the catalytic metal-containing zeolite comprises up to 10 wt. % of the metal or metal oxide,

7. The method of claim 6, wherein the catalytic metal is gallium (Ga), nickel (Ni), copper (Cu), iron (Fe), cobalt (Co), chromium (Cr), zinc (Zn), molybdenum (Mo) or oxides thereof, or combinations thereof.

8. The method of claim 7, wherein the metal or metal oxide comprises Ga or Mo.

9. The method of claim 1, wherein the zeolite is ZSM-5, a H-ZSM-5, a hollow ZSM-5, a hollow H-ZMS-5.

10. The method of claim 1, wherein the reaction conditions further include a pressure of 0.5 to 5 bar, and a weight hourly space velocity (WHSV) of 0.5 h⁻¹ to 20 h⁻¹, 1 h⁻¹ to 10 h⁻¹.

11. The method of claim 1, wherein the C₃ alcohol is isopropanol or a mixture of isopropanol and propanol.

12. The method of claim 1, wherein the ratio of C₃ alcohol to CO₂ is 0≤5.

13. The method of claim 1, wherein the metal or metal oxide comprises Ga.

14. The method of claim 13, wherein the reactant feed further comprises a carrier gas, and wherein the carrier gas is helium, argon, nitrogen, or carbon dioxide.

15. The method of claim 1, wherein the reactant feed consists essentially of or consists of the C₃ alcohol and CO₂.

16. The method of claim 1, wherein the reactant feed does not include any other alcohol other than a C₃ alcohol.

17. The method of claim 1, wherein the reactant feed comprises carbon dioxide (CO₂); and
wherein the ratio of C₃ alcohol to CO₂ is 0.1≤5.

18. The method of claim 17, wherein the combined selectivity of benzene, toluene, and xylenes is at least 30%.

19. (canceled)

20. The method of claim 1, wherein the zeolite is ZSM-5 zeolite or a H-ZSM-5 zeolite, wherein the catalytic metal-containing zeolite comprises up to 10 wt. % of the metal or metal oxide, and wherein the catalytic metal is gallium (Ga), nickel (Ni), copper (Cu), iron (Fe), cobalt (Co), chromium (Cr), zinc (Zn), molybdenum (Mo) or oxides thereof, or combinations thereof.

21. The method of claim 20, wherein the zeolite is a hollow ZSM-5 zeolite or a hollow H-ZSM-5 zeolite.
